(19) United States
(12) Patent Application Publication
(10) Pub. No.: US 2022/0339269 A1
(43) Pub. Date: Oct. 27, 2022

(54) PANCREATIN MICROCAPSULES

(71) Applicant: AVVA PHARMACEUTICALS LTD., Mesa Geitonia, Limassol (CY)

(72) Inventors: Artem Sergeevich TSVETKOV, Kirov (RU); Pavel Valeryevich SEVODIN, Biysk (RU)

(21) Appl. No.: 17/640,918

(22) PCT Filed: Sep. 3, 2020
(86) PCT No.: PCT/RU2020/000464

§ 371 Date: Mar. 7, 2022

(30) Foreign Application Priority Data
Sep. 9, 2019 (RU) 2019128267

(51) Int. Cl. A61K 38/54; A61K 9/50; A61K 9/48

(52) U.S. Cl. CPC A61K 38/54; A61K 9/5089; A61K 9/5031; A61K 9/501; A61K 9/4866; A61K 9/4858

(57) ABSTRACT

The invention relates to a pharmaceutical composition of the cores of microgranules containing pancreatin, cetyl alcohol, poloxamer 407 in predetermined quantities, the production method, as well as the production of the water-based enteric-coated microgranules. Furthermore, the resulting oral dosage form does not contain residual acetone. The technical result is in achieving higher stability of the cores and the enteric-coated microgranules, respectively, while maintaining the good solubility of the enteric-coated microgranules, which allows the application of the claimed pancreatin microgranules for the preparation of safe and non-toxic drugs for the treatment of digestive disorders.

The invention is relates to the area of medicine and concerns a pancreatin-based enzyme product in an oral dosage form of enteric-coated microgranules.

In most detail, the present invention describes a pharmaceutical composition that is produced as cores of microgranules with pancreatin, cetyl alcohol, and poloxamer 407 in pharmaceutically effective amounts.

The invention is relative to a production method of the indicated pharmaceutical composition, as well as to the production of microgranules based on a pharmaceutical composition, coated with a water-based enteric coating.

The enteric-coated microgranules are obtained by the method according to the invention and are used as a drug for the treatment of exocrine (enzymatic) pancreatic insufficiency in replacement therapy for children and adults due to a decrease in pancreatic enzyme activity because of the disturbances in production, secretion regulation, and pancreatic enzymes delivery or their increased destruction in the intestinal lumen, which is caused by various diseases of the gastrointestinal tract, most common in cystic fibrosis, chronic pancreatitis, after pancreatic surgery, after gastrectomy; pancreatic cancer; partial resection of the stomach (for example, Billroth II).

### BACKGROUND OF THE INVENTION

Pancreatin is known to be a mixture of various physiologically active digestive enzymes such as lipase, amylase, and protease. Pancreatic lipases, amylases, and proteases are active digestive enzyme supplements in the treatment of different pathological conditions, such as pancreatic exocrine insufficiency.

A digestive enzyme is a pancreatic enzyme and refers to any of the types of enzymes present in pancreatic secretions, e.g., amylase, lipase, protease, or mixtures thereof, or any pancreatic extract with enzymatic activity, such as pancreatin. Classes of digestive enzymes, proper for application in the present invention, may at least include lipases, amylases, and proteases.

Pancreatin or digestive enzymes are usually made in the form of tablets, capsules, or granules, which must satisfy a number of drug requirements. Of these, there are such requirements, according to which, tablets, capsules or granules should protect the contained enzymes inside from degradation during passage through the stomach at pH=1.0 and in the upper intestine at pH=5.0 or pH=6, and also dissolve when it enters the small intestine at pH=6.0, releasing active enzymes in the required amount and be safe for use in patients, in particular not to be toxic.

The maintenance of enzyme activity during ingestion and rapid release in the intestine, particularly stability and solubility are important characteristics of the product. In many studies, it was found that granules, microgranules, or pellets, for example, are the most effective forms of enzyme delivery to the intestine.

There are many known methods for producing granules or pellets, which include the following steps: preparing an extrudable mixture, extruding cores of pellets, drying the obtained cores of pellets, applying the enteric coating, and drying the pellets. For instance, enteric-coated pancreatin pellets with 65-85 wt % are described in European patent EP 0583726, especially with 75-80 wt % of pancreatin, the volume density of which is from 0.6 g/ml to 0.85 g/ml, and mainly containing pancreatin, polyethylene glycol 4000 and low viscosity paraffin, containing per 100 weight parts of pancreatin: 15-50 wt. part. (especially 20-30 wt. part.) of polyethylene glycol 4000 and 1.5-5 wt. part. (especially 2-3 wt. wt. part.) of low viscosity paraffin, characterized by a spherical or ellipsoidal shape, while the diameter of the sphere or the small axis of the ellipse is from 0.7 to 1.4 mm, preferably from 0.8 to 1.2 mm, and characterized by a particle size distribution in which at least 80% of the pancreatin micropellets is characterized by the ratio of the small axis of the ellipse to the major axis of the ellipse in the range from 1:1 to 1:2.

The disadvantage of these enzyme products is the presence in the ingredient composition of low viscosity paraffin and mineral oil, particularly petroleum jelly, which is critical since it is not currently recommended to prescribe mineral oils to pregnant women or infants.

The patent RU 2440101C2 describes a pharmaceutical composition comprising an oral dosage form of pancreatin with the enteric coating that includes a film-forming agent, cetyl alcohol and triethyl acetate as a plasticizer, and at least one anti-sticking agent.

The closest in technical essence is the patent RU 2408364 C2, which describes a method for producing and application of the cores of pancreatin micropellets. Also, the enteric coating of pancreatin micropellets consisting of a film-forming agent and a plasticizer is described.

In a known manner it is possible to obtain the cores of pancreatin micropellets containing from 70 to 90 wt % of pancreatin, from 10 to 30 wt % of at least one pharmaceutically acceptable binding agent, and up to 5 wt % of at least one pharmaceutically acceptable inert filler.

The disadvantages of this production method are the presence of traces of acetone in the resulting micropellets, as well as the lack of stability of the enzymes under conditions at pH=1-6.

The appearance of residual acetone in the composition obtained according to the above-described method for pancreatin micropellets makes this product unsafe for the patient, because acetone has a 3rd class of toxicity, and can cause damage to some organs (e.g., skin and lungs). Despite the fact that the amount of residual acetone in the composition of such micropellets is poor, however prolonged or even lifelong application of multiple doses (during the day) of these micropellets in the treatment of a number of diseases, especially cystic fibrosis, can lead to additional health threats.

Therefore, the objective of the present invention was to provide a new pharmaceutical composition for producing an enzyme product with pancreatin in an oral dosage form of microgranules with low toxicity, i.e. without residual amounts of acetone, with high stability and solubility, without restrictions of application in patients of all ages and from all specific groups, due to the qualitative and quantitative composition of the ingredients in an oral dosage form with the enteric coating.

One of the implementations of the described invention is an oral dosage form of pancreatin microgranules with the enteric coating.

The problem was solved thanks to the new composition of the core of microgranule, which contains pancreatin, cetyl alcohol, poloxamer 407 in predetermined quantities, a new method for production of such cores, as well as the production of microgranules themselves with the enteric water-based coating. Moreover, the resulting oral dosage form does not contain residual amounts of acetone and synthetic oils.

### ESSENCE OF THE INVENTION

In a more detailed preferred implementation of the invention, an oral dosage form (the core of microgranule) for the preparation of a drug for the treatment of digestive disorders associated with pancreatic exocrine insufficiency, dyspepsia, pancreatitis, cystic fibrosis, type I diabetes and/or type II diabetes, containing from 95.6 to 98.0 wt % of pancreatin, from 1.0 to 2.3 wt % of cetyl alcohol, and from 1.0 to 2.10 wt % of poloxamer 407. Herewith the cores obtained after the stage of spheronization have the following size: d (core diameter)=1.0-1.2 mm, 1 (core length)=0.8-2.0 mm.

Poloxamer 407 is a triblock copolymer comprising a central hydrophobic polypropylene glycol (PPG) block between two hydrophilic polyethylene glycol (PEG) blocks.

Also, the present invention is related to a method of producing the pharmaceutical composition due to a binding agent that is necessary for the formation of a core of a microgranule. First of all, particularly, prepare a mixture of the binding agent consisting of three components:ethyl alcohol, cetyl alcohol, poloxamer 407, in the following range of ratios from 1:0.11:0.11 to 1:0.13:0.13, subject to certain technological conditions, e.g. while maintaining the process temperature in the range from 40 to 45° C. Pancreatin is mixed with the resulting binder mixture in the presence of a solvent, in particular, ethyl alcohol in predetermined optimal amounts. Furthermore, in a preferred implementation of this method, the production stages of the pharmaceutical composition are carried out in a strict sequence, which involves adding ethanol to pancreatin in effective amounts, followed by introducing into the system a preformed three-component binding agent.

After obtaining a homogeneous mixture, carry out the molding and spheronization of the cores of microgranules in the presence of ethyl alcohol. Further, carry out the drying of the cores of microgranules, during which ethyl alcohol is removed, and maintain the temperature at 34° C. Pancreatin microgranule is obtained by applying an enteric coating solution on a core.

In one implementation of the invention, the enteric coating comprises water, macrogol 4000, talc, an emulsion of simethicone, a suspension of methacrylic acid and ethyl acrylate copolymer in a ratio of 1:1. The pharmaceutical composition and/or microgranules are used in a dosage form suitable for oral administration with the possibility of preparing a drug intended for the treatment of digestive disorders associated with pancreatic exocrine insufficiency, dyspepsia, pancreatitis, cystic fibrosis, type I diabetes and/or type II diabetes.

The pharmaceutical composition and/or microgranules are used in a dosage form suitable for oral administration with the possibility of preparing a medicament intended for the treatment of digestive disorders associated with pancreatic exocrine insufficiency, dyspepsia, pancreatitis, cystic fibrosis, type I diabetes and/or type II diabetes. Moreover, mixing pancreatin with ethyl alcohol in a strict sequence, according to which, firstly, pancreatin is taken, then ethyl alcohol is added, after which the three-component binding agent is introduced into the system, results in homogenicity of the obtained suspension. It was found that the gradual (discrete) introduction of the binding agent during the preparation of the suspension makes it highly homogeneous.

Moreover, the functional use of cetyl alcohol (fatty alcohol) as a binding agent, promotes compaction during the formation of the cores of microgranules and increases adhesion between particles.

Adding poloxamer 407, which acts as a binding and solubilizing agent, strengthens the core of pancreatin microgranules and increases the dissolution of the active pharmaceutical ingredient, i.e. pancreatin.

The research results provided below showed that the claimed invention allows producing microgranules of pancreatin with high resistance to the action of gastric acid at pH=4.0, as well as with a rather high dissolution rate at pH=6.0 (for no more than 30 minutes). The enteric coating is applied to an oral dosage form of a drug with pancreatin, which must be delivered to the gastrointestinal tract to an area where the pH is greater than in the stomach. The claimed composition of the enteric coating in the present invention is water-based, which in turn provides good solubility at pH=6.0, without loss of enzymatic activity of a core of microgranule, and absence of toxicity, e.g. compared with enzyme products where acetone residues are present.

According to this invention, it was unexpectedly found that the cores of pancreatin microgranules, which are prepared according to the claimed method, are suitable for applying the enteric coating and have high stability while maintaining enzymatic activity. It was also discovered that the method for producing the cores of microgranules described in the invention is more effective in terms of the safety of the components used compared to other known methods. Particularly, the suggested enteric coating of the cores of pancreatin microgranules, unlike other known compositions of the enteric coating, does not contain an organic solvent such as toxic acetone. It was unexpectedly found that the composition of the enteric coating on a water basis is not inferior in its properties to the composition of the enteric coating on the basis of acetone. Further, the applying of the enteric water-based coating to pancreatin microgranules positively affects product stability. The storage stability of the cores of pancreatin microgranules shows good results thanks to the use of predetermined proportions of cetyl alcohol and poloxamer 407 in the composition of the cores.

According to the invention, it was unexpectedly found that the pharmaceutical composition as part of the enteric-coated core of microgranule, comprising water, macrogol 4000, talc, an emulsion of simethicone, a suspension of methacrylic acid and ethyl acrylate, is stable in gastric acid (at pH=1.0), as well as in the upper intestine (at pH=6.0).

In another implementation of the invention, the enteric coating of a microgranule may be water-based, in particular, contain water, triethyl citrate, talc, simethicone, a mixture of methacrylic acid and ethyl acrylate in pharmaceutically effective amounts.

To obtain oral dosage form of the medicinal product microgranules should be formed and spheronised from the resulting mixture with ethyl alcohol, the procedure should be followed with core drying under conditions providing complete elimination of used ethanol. Molding is an extrusion process of the resulting mixture of pancreatin and the binding agent. According to the invention, the optimum drying temperature of the formed pancreatin cores is maintained at about 34° C. This temperature regime ensures better preservation of the enzyme components.

Also, the invention is related to the method for producing the pancreatin microgranules with the enteric coating containing water, triethyl acetate, talc, simethicone, a mixture of methacrylic acid and ethyl lactylate in pharmaceutically acceptable amounts. Microbeads also do not contain residual acetone.

The claimed invention allows obtaining pancreatin microgranules with high resistance to the action of gastric acid at pH=1.0, as well as to rapid dissolution at pH 6.0 for not longer than 30 minutes.

It was unexpectedly found that the composition of the enteric coating on a water basis is not inferior in its properties to the composition of the enteric coating on the basis of acetone. Unexpectedly, it was found that the composition of the enteric coating on a water basis is not inferior in its properties to the composition of the enteric coating on the basis of acetone. Further, the applying of the enteric water-based coating to pancreatin microgranules positively affects product stability. The storage stability of the cores of pancreatin microgranules shows good results.

When applying the enteric coating on microgranules, as described in the present invention, water was used as the main solvent. This can be explained by the following reasons: no risk of exposure of hazardous factors such as toxic effects of vapors, explosion and fire hazard; non-explosion-proof equipment and less expensive fireproof premises; no need for investigation on the presence of residual solvents in the product, and no toxicity, respectively.

Therefore, in the present invention, an optimal ratio of the main components for the cores of microgranules and a specific sequence of the introduction of the binding agent are achieved, which leads to a pharmaceutical composition that allows the digestive enzyme pancreatin to be in a stable state, providing effective release in the intestine with minimal loss of activity under typical storage conditions.

The technical result of the present invention is to achieve higher stability of the cores and the enteric-coated microgranules, respectively, while maintaining good solubility of the microgranules with the enteric coating, which allows the application of the claimed pancreatin microgranules for producing safe and non-toxic drugs intended for the treatment of digestive disorders due to pancreatic exocrine insufficiency, dyspepsia, pancreatitis, type I and/or type II diabetes. The technological conditions of the method for producing a core and microgranules with the water-based enteric coating make it possible to achieve high solubility of a drug, storage stability without loss of enzymatic activity, as well as to ensure safe application for patients of all age groups needed treatment of digestive disorders.

Tests of pancreatin microgranules obtained by this method have shown that a higher lipase content is maintained compared to other known pancreatin micropellets in which other binding agents are used.

Therefore, the pancreatin microgranules described in this invention are highly resistant to gastric acid and have a protective ability, e.g. a protective ability at pH=1 and/or pH=6.

According to the invention, the drying temperature of the pancreatin microgranules was maintained in the range of 35-50° C. to ensure better preservation of the enzyme components.

### THE IMPLEMENTATION OF THE INVENTION

### Example 1 (According to the Invention)

An example illustrates the preparation of a pharmaceutical composition containing pancreatin in the presence of an ethyl alcohol solvent with the possibility of obtaining the cores of microgranules.

Initially, a solution of the binding agent is prepared.

According to one implementation of the invention, ethyl alcohol is fed into a manufacturing vessel equipped with a stirrer and a heated jacket at 40-45° C. Then, with stirring, cetyl alcohol (in powder), poloxamer 407 (in powder) are added at a ratio of ethyl alcohol:cetyl alcohol:poloxamer 407-1:0.11:0.11, respectively. Therefore, a ternary mixture of the binding agent is obtained.

According to one implementation of the invention, ethyl alcohol is fed into a manufacturing vessel equipped with a stirrer and a heated jacket at 40-45° C. Then, with stirring, cetyl alcohol (in powder) and poloxamer 407 (in powder) are added at a ratio of ethyl alcohol:cetyl alcohol:poloxamer 407-1:0.12:0.12, respectively.

According to another implementation of the invention, ethyl alcohol is fed into a manufacturing vessel equipped with a stirrer and a heated jacket at 40-45° C. Next, with stirring, cetyl alcohol (in powder) and poloxamer 407 (in powder) are added at a ratio of ethyl alcohol:cetyl alcohol:poloxamer 407-1:0.13:0.13, respectively.

According to one implementation of the invention, pancreatin is loaded into the mixer-granulator, then ethanol is added to improve wetting properties, then the prepared binding agent is loaded into the moistened mixture taking into account the above ratios at ambient temperature. After each supply of one of the components, the mixture is thoroughly stirred. The defined consistent supply of ingredients, in particular, pre-mixing pancreatin with ethyl alcohol, ensures uniform dissolution of the components and more efficient interaction of the components in the cores of microgranules.

The resulting mixture was loaded for extrusion using dies with a hole size of 1.0 mm and with a controlled temperature of granules at 30° C. The resulting cores with a diameter from 1.0 to 1.2 mm and length from 0.8 to 2.0 mm are fed to the spheronization step, which is carried out in the presence of an ethyl alcohol solvent with a concentration of 55-96 wt %, with a ratio of pancreatin to ethyl alcohol of 1:0.38.

During the method implementation, the cores of microgranules are obtained, the composition of which is given in the Table.

TABLE 1
The composition of the cores of microgranules, in wt %

| | | | | | | | |
|---|---|---|---|---|---|---|---|
| 1 | Pancreatin | 95.6 | 96.00 | 96.55 | 96.85 | 97.50 | 98.00 |
| 2 | Cetyl alcohol | 2.30 | 2.00 | 1.50 | 1.57 | 1.25 | 1.00 |
| 3 | Poloxamer 407 | 2.10 | 2.00 | 1.95 | 1.57 | 1.25 | 1.00 |
| Core | Diameter (Ø) of the cores of | 1.00 | 1.00 | 1.20 | 1.10 | 1.15 | 1.10 |
| sizes | microgranules, mm | | | | | | |
| | The length (l) of the cores of | 0.80 | 1.00 | 1.30 | 1.15 | 1.20 | 2.00 |
| | microgranules, mm | | | | | | |

After spheronization, the obtained cores of microgranules are subjected to drying, which is carried out at a temperature of 34° C., humidity 2-5 wt %. The dried cores are fed for the next applying of the enteric coating solution.

The enteric coating solution is prepared by mixing of the ingredients:water, macrogol 4000, talc, a 30% emulsion of simethicone, a suspension of methacrylic acid and ethyl acrylate copolymer (1:1) (dispersion 30%) at a ratio of 1:0.03:0.14:0.004:1.04. Application of the prepared solution on the cores of microgranules is carried out by spraying on granules preheated to 35° C. with a ratio of microgranules to the resulting solution of 1:1.57. At the end of the step of applying the enteric coating solution, the obtained microgranules are dried while maintaining the temperature in the range from 35° C. to 50° C.

### Example 2 (Comparative by the Method of Producing the Cores of Microgranules)

The method for producing the cores of pancreatin microgranules, as in Example 1, with the difference that, first of all, ethyl alcohol is loaded into the mixer-granulator, and pancreatin is added in portions, then the binding agent is added in the ratios indicated in Example 1. After each supply of one of the components, the mixture is thoroughly mixed for 15 minutes.

### Example 3 (Comparative by the Method of Producing the Cores of Microgranules)

The method for producing the cores of pancreatin microgranules, as in Example 1, with the difference that the prepared mixture of pancreatin with the binding agent is loaded into the hopper of a molding machine. The suspension is granulated using dies with hole sizes of 1.0 mm with a controlled drying temperature of the cores at 28° C.

### Example 4 (Comparative by the Method of Producing the Cores of Microgranules)

The method for producing the cores of pancreatin microgranules, as in Example 1, with the difference that the prepared mixture of pancreatin with the binding agent is loaded into the hopper of a molding machine. The suspension is granulated using dies with a hole size of 1.0 mm with a controlled drying temperature of the cores at 41° C.

### Example 5 (Comparative by the Method of Producing the Cores of Microgranules)

The method for producing the cores of pancreatin microgranules, as in Example 1, with the difference that the prepared mixture of pancreatin with the binding agent is loaded into the hopper of a molding machine. The suspension is granulated using dies with a hole size of 1.0 mm with a controlled drying temperature of the cores at 38° C.

### Example 6 (According to the Invention)

The method of producing pancreatin microgranules according to Example 1 with the difference that the ratio of the ingredients in the solution of the enteric coating—water, macrogol 4000, talc, a 30% emulsion of simethicone, a suspension of methacrylic acid and ethyl acrylate copolymer (1:1) (dispersion 30%)— is in a ratio of 1:0.03:0.16:0.004:1.03. The resulting solution was stirred for 15 minutes.

### Example 7 (Comparative with the Prototype)

The method of producing micropellets according to the prototype where acetone was used as a solvent in the preparation of the enteric coating.

1. Preparation of Pancreatin Micropellets:

1.59 kg of pancreatin was mixed with 0.25 kg of polyethylene glycol 4000 in a mixer, thoroughly moistened with 0.25 kg of 2-propanol. The resulting mixture was molded with an inner diameter of 1.0 mm. During pressing, the temperature was below 50° C. The resulting 1.46 kg of pancreatin after molding, in equal portions, were sent to the spheronization step for obtaining the cores of micropellets. During spheronization, about 13.5 g of 2-propanol was added. After drying at a temperature in the range from 35 to 50° C. for 12 h, pancreatin microgranules were sorted by sieving on sieves.

2. Applying of the Enteric Coating:

The coating solution was prepared by adding with stirring 231.4 g of hydroxypropyl methylcellulose phthalate, 12.85 g of triethyl citrate, 4.89 g of cetyl alcohol, and 5.55 g of dimethicone 1000 to 2000 g of acetone at room temperature. The coating was applied until film formation by spraying the resulting solution on pancreatin micropellets. The temperature of the cores of micropellets during coating was maintained in the range from 37 to 43° C. Then the obtained micropellets were dried at a temperature in the range from 35 to 50° C. for 12 hours.

Stability Test of Pancreatin Microgranules Obtained According to the Present Invention

For the cores of pancreatin microgranules, obtained by the methods that are described in Examples 1-7, the stability tests were carried out under conditions simulating the environment of the stomach at pH=1.0 and the environment of the upper intestine at pH=6.0. The determination of the amount of active substance, which for a certain period of time must be released into the dissolution medium from a solid dosage form, was carried out by the biochemical method, in accordance with the SP XIII GPM 1.4.2.0014.15 “Dissolution for solid pharmaceutical dose forms” at pH=1.0 and pH=6.0.

The Dissolution test is carried out in two steps.

Step 1 (acidic, i.e. pH=1.0) using an impeller mixer.

4M sodium hydroxide solution. 16.0 g of sodium hydroxide is placed in a 100 ml volumetric flask, dissolved in 80 ml of water, the temperature in the range of 37±0.5° C. After cooling the solution to room temperature, the solution is diluted to the necessary volume and mixed. The test time is 120 minutes.

Step 2 (alkaline, i.e. pH=6.0) using an impeller mixer.

A phosphate buffer solution at pH=6.0, in which 2.0 g of sodium chloride and 9.2 g of monosubstituted potassium phosphate are placed in a 1000 ml volumetric flask and dissolved in approximately 950 ml of water. The pH of the solution is adjusted potentiometrically to 6.0 using a 4M sodium hydroxide solution, the solution is diluted to the necessary volume, temperature 37±0.5° C. The dissolution process lasts for 30 minutes.

The average values obtained during the testing of samples at pH=1.0 for 120 minutes and at pH=6.0 for 30 minutes are shown in Tables 2-4.

The dissolution stability characteristics under conditions simulating the gastric environment of the prepared samples are given as the percentage of residual lipolytic activity after incubation in terms of the actual lipolytic activity of the samples tested before incubation.

According to the above GPM “Dissolution for solid pharmaceutical dose forms”, the amount of active substance released into the dissolution medium when released in the stomach should not exceed 10% of the claimed content of pancreatin; in the environment of the upper intestine should be at least 75% of the claimed content of pancreatin.

Lipolytic activity was determined by the biochemical method, in comparison with the specific activity of pancreatin enzymes of a standard sample. Lipolytic activity is determined by comparing the rate at which a suspension of pancreatin microgranules hydrolyzes an olive oil emulsion substrate with the rate at which a suspension of a standard pancreatin (lipase) sample hydrolyzes the same substrate under the same conditions.

Comparison of the dissolution stability indicators of pancreatin microgranules under conditions simulating the environment of the stomach and the environment of the upper intestine according to Examples 1 and 2.

TABLE 2
The dissolution stability of pancreatin microgranules
under conditions simulating the environment of the stomach
(at pH = 1.0) and upper intestine (at pH = 6.0)

| | |
|---|---|
| Samples of microgranules | |
| prepared according to | Stability of samples |

| examples of the invention | at pH = 1.0, % | At pH = 6.0, % |
|---|---|---|
| The core of microgranules | 4.6 | 93.7 |
| according to Example 1 | | |
| The core of microgranules | 6.8 | 83.8 |
| according to Example 2 | | |

The results of the Dissolution test indicates that the gastric acid resistance of the composition according to Example 1 at pH=1.0 and pH=6.0 exceeds the stability indices of the composition obtained in Example 2. The stability of the samples at pH=1 should be no more than 10%, which is a control option.

In accordance with the general article of pharmacopoeia of the State Pharmacopoeia XIII, OFS, 1.4.2.0014.15 “Dissolution for solid dosage forms”, the amount of active substance released into the dissolution medium when released in the stomach should be no more than 10% of the claimed content of the pancreatin; in the environment of the upper intestine must be at least 75% of the claimed content of pancreatin.

Comparison of the dissolution stability indicators of pancreatin microgranules under conditions simulating the environment of the stomach and the environment of the upper intestine according to Examples 3, 4, and 5.

TABLE 3
Dissolution stability of pancreatin microgranules under conditions
simulating the environment of the stomach (pH = 1.0)
and the environment of the upper intestine (pH = 6.0)

| Sample | pH 1.0% | pH 6.0, % |
|---|---|---|
| The core of microgranules | 6.1 | 87.3 |
| according to Example 3 | | |
| The core of microgranules | 7.0 | 84.6 |
| according to Example 4 | | |
| The core of microgranules | 6.4 | 89.5 |
| according to Example 5 | | |

The obtained results of comparison of the dissolution stability indicators of pancreatin microgranules under conditions simulating the stomach and upper intestine environment, for compositions prepared according to Examples 3, 4 and 5, when comparing different temperatures of drying, clearly show the advantage of the selected temperature at 34° C. for drying the cores of microgranules after spheronization.

Comparison of the results of gastric acid resistance tests at pH=1.0 and pH=6.0 of the composition prepared according to Examples 1 and 2 and the prototype.

TABLE 4
Dissolution stability of pancreatin microgranules under conditions
simulating the environment of the stomach (pH = 1.0)
and the environment of the upper intestine (pH = 6.0)

| Sample | pH 1.0, % | pH 6.0, % |
|---|---|---|
| The core of microgranules | 4.6 | 93.7 |
| according to Example 1 | | |
| The core of microgranules | 6.8 | 83.8 |
| according to Example 2 | | |
| The core of microgranules | 5.9 | 86.8 |
| according to the prototype | | |

The results of comparing the stability indicators of pancreatin microgranules when dissolved under conditions simulating the environment of the stomach and the environment of the upper intestine, the compositions made according to Examples 1 and 2 show the advantages of the proposed method for producing the pharmaceutical composition of the cores of microgranules compared to the prototype composition.

Therefore, it was found that the tested samples of pancreatin microgranules are resistant to gastric acid at pH=1.0, and their activity is not less than:

4.6% (even more preferred value) for the sample prepared according to Example 1;

7.0% (preferred value) for the sample prepared according to Example 4;

6.8% (preferred value) for the sample prepared according to Example 2;

6.4% (more preferred value) for the sample prepared according to Example 5;

6.1% (more preferred value) for the sample prepared according to Example 3;

from a predetermined lipolytic activity of the pancreatin standard.

The pancreatin microgranules in the present invention are stable when released in the upper intestine at pH=6.0, and the yield is not less than:

93.7% (even more preferred value) for the sample prepared according to Example 1;

89.5% (more preferred value) for the sample prepared according to Example 5;

87.3% (more preferred value) for the sample prepared according to Example 3;

84.6% (preferred value) for the sample prepared according to Example 4;

83.8% (preferred value) for the sample prepared according to Example 2;

from a predetermined lipolytic activity of the pancreatin standard.

The storage stability of the cores of pancreatin microgranules shows good results.

Unexpectedly, it was found that the enteric water-based coating is not inferior in its properties to an acetone-based coating. Subsequently, applying the enteric coating on pancreatin microgranules in the presence of an aqueous solvent positively affects the storage stability of the pharmaceutical composition at control points.

TABLE 5
The storage stability of the cores of pancreatin
microgranules within 90 and 180 days

| | Stability | Stability | Stability | Stability |
|---|---|---|---|---|
| | at pH = | at pH = | at pH = | at pH = |
| | 1.0, %, | 6.0, %, | 1.0, %, | 6.0, %, |
| Sample | 90 days | 90 days | 180 days | 180 days |
| The core of microgranules | 4.1 | 89.3 | 4.9 | 90.5 |
| according to Example 1 | | | | |
| The core of microgranules | 6.8 | 80.7 | 6.5 | 81.8 |
| according to Example 2 | | | | |
| The core of microgranules | 5.2 | 84.7 | 5.1 | 83.4 |
| according to Example 3 | | | | |
| The core of microgranules | 6.3 | 82.2 | 6.0 | 80.9 |
| according to Example 4 | | | | |
| The core of microgranules | 5.6 | 85.7 | 5.3 | 87.0 |
| according to Example 5 | | | | |
| The core of microgranules | 5.4 | 82.1 | 5.9 | 89.6 |
| according to Example 6 | | | | |
| The core of microgranules | 5.9 | 88.4 | 6.2 | 87.1 |
| according to the prototype | | | | |

1. A pharmaceutical composition for the preparation of a drug for the treatment of digestive disorders associated with pancreatic exocrine insufficiency, dyspepsia, pancreatitis, cystic fibrosis, type I diabetes and/or type II diabetes, comprising:
Pancreatin in the amount from 95.6 wt % up to 98.0 wt %,
Cetyl alcohol in the amount from 1.0 wt % up to 2.3 wt %,
Poloxamer 407 in the amount from 1.0 wt % up to 2.1 wt %,
in the form of the cores of microgranules with the following sizes:
d (core diameter)=1.0-1.2 mm, 1 (core length)=0.8-2.0 mm.

2. A method of production of the pharmaceutical composition according to claim 1, comprising the steps of:
a) preparation of a mixture of a binding agent, consisting of three components, which are ethyl alcohol, cetyl alcohol, poloxamer 407 in the following ratios 1:0.11:0.11-1:0.13:0.13, besides, the process is carried out at T=40-45° C.
b) mixing pancreatin with ethyl alcohol, followed by the addition of the binding agent obtained in step (a).
c) molding and spheronization of the cores of microgranules from the resulting mixture in the presence of ethyl alcohol;
d) drying the cores of the microgranules with the possibility of removing ethyl alcohol.

3. The method according to claim 1 where step (d) is carried out at a temperature of not more than 34° C.

4. Pancreatin microgranule containing the pharmaceutical composition according to claim 1 and the enteric coating comprising water, macrogol 4000, talc, an emulsion of simethicone, a suspension of methacrylic acid and ethyl acrylate copolymer in a ratio of 1:1, in a dosage form suitable for oral administration.

5. The application of the cores of pancreatin microgranules according to claim 1 for the preparation of a drug for the treatment of digestive disorders associated with pancreatic exocrine insufficiency, dyspepsia, pancreatitis, cystic fibrosis, type I diabetes and/or type II diabetes.

6. The application of pancreatin microgranules according to claim 4 for the preparation of a drug for the treatment of digestive disorders associated with pancreatic exocrine insufficiency, dyspepsia, pancreatitis, cystic fibrosis, type I diabetes and/or type II diabetes.
